(19) United States
(12) Patent Application Publication
(10) Pub. No.: US 2011/0224337 A1
(43) Pub. Date: Sep. 15, 2011

(54) PROCESS FOR PREPARING AN N-ALKYLLACTAM WITH IMPROVED COLOR QUALITY

(75) Inventors: Yoshihisa Fujii, London (GB); Kanno Hiroyasu, Tokyo (JP)

(73) Assignee: BASF SE, Ludwigshafen (DE)

(21) Appl. No.: 13/130,853

(22) PCT Filed: Nov. 18, 2009
(86) PCT No.: PCT/EP2009/065387

§ 371 Date: May 24, 2011

(30) Foreign Application Priority Data
Nov. 24, 2008 (EP) 08169740.1
Apr. 24, 2009 (EP) 09158734.5

(51) Int. Cl. C08K 5/3415; C07D 225/02; C07D 223/10; C07D 211/76; C07D 207/50; C08K 5/3435; C08K 5/3412

(52) U.S. Cl. 524/98; 540/451; 540/540; 546/244; 548/558; 524/104; 524/99; 524/86

(57) ABSTRACT

The present invention relates to a process for purifying an N-alkyllactam which comprises contacting N-alkyllactams with carbon. The present invention also relates to mixtures comprising poly(vinylidene fluodride) (PVDF) and N-alkyllactams, in which the N-alkyllactams have been purified by contacting the N-alkyllactam as well as a method for their production. The present invention also relates to the use of said mixtures in processing PVDF for applications in which improved color is a quality requirement.

The present invention relates to a process for purifying an N-alkyllactam which comprises contacting N-alkyllactams with carbon. The present invention also relates to mixtures comprising poly(vinylidene fluodride) (PVDF) and N-alkyllactams, in which the N-alkyllactams have been purified by contacting the N-alkyllactam as well as a method for their production. The present invention also relates to the use of said mixtures in processing PVDF for applications in which improved color is a quality requirement.

Purification processes for N-alkyllactams are known. The purification of N N-alkyllactams can be affected, for example, by fractional distillation (including multiple distillation, as described in JP 06 228 088 (Mitsubishi Kasei Corp.)) or by extraction. Other or additional purification steps may be treatment with ion exchangers as described, for example, in EP-A-1 038 867 (BASF AG), or with solid adsorbents (U.S. Pat. No. 4,501,902), such as aluminum oxide analogously to WO-A-2005/092851 (Lyondell L. P.). N-alkyllactams can also be purified in the presence of acids such as toluenesulfonic acid (described, for example, JP 11 071 346 (Tonen Corp.)) or phosphoric acid (described, for example, in JP 2028148 (Ouchi Shinko Chem.)) during distillation. Other advantageous additives during the preparation and/or distillation may be alkali metal, alkaline earth metal or ammonium borohydrides, as disclosed, for example, in U.S. Pat. No. 4,885,371 (GAF Chemicals Corp.), oxidizing agents such as potassium permanganate, sodium perborate or potassium dichromate as described in JP 72 22 225 (Teijin Ltd.), or sodium hydroxide as described in U.S. Pat. No. 2,964,535 (Monsanto Chemicals).

In addition, JP-A-2001 089 446 (Mitsubishi Chem. Corp.), teaches that clean NMP (N-Methylpyrrolidone) with low color can be obtained when the amounts of hydrogen and oxygen do not exceed limiting values of 0.01 mol % and 0.002 mol % based on the pyrrolidone content during distillation. According to JP 62 79 401 (Mitsubishi Kasei Corp.), colorless N-methylpyrrolidone can also be obtained by thermal treatment (heating at 150-250° C.) and subsequent distillation.

Other N-alkyllactams such as N-alkylpiperidones and N-alkylcaprolactams can be purified in analogous ways.

Poly(vinylidene fluodride) (PVDF) is the addition polymer of 1,1-difluoroethene, also known as vinylidene fluoride.

PVDF is a semicrystalline polymer which is usually polymerized in emulsion or suspension using free-radical initiators.

PVDF combines the characteristic resistance of fluropolymers to harsh chemical, thermal, ultraviolet, weathering and oxidizing environments with other unique properties such as a high polarity, a high dielectric constant as well as an excellent piezoelectric and pyroelectric activity.

Because of these properties PVDF is used in many applications, e.g. in wire and cable products, electronic devices, as a weather resistant binder for exterior architectural finishes.

The polymer is readily melt processed using conventional moulding or extrusion equipment of cast from solutions to form membranes and films. Finishes are deposited from dispersions using specific solvents.

When processing PVDF from or in solution usually a solvent with a high polarity is selected. For many applications N-alkyllactams, such as N-Methyl-pyrrolidone (NMP), or formamides, such as dimethyl formamide of sulfoxides, such as dimethyl sulfoxide, among others have been suggested.

The use of N-alkyllactams as a processing solvent for PVDF has a decisive setback. JP-A1-10310795 discloses that solutions of PVDF in N-alkyllactams tend to discolor after a short while due to a small amount of impurities contained in N-alkyllactams making these solutions unsuitable for use in high end applications, such as electronics or coatings. To avoid these problems, JP-A1-10310795 teaches a method for dissolving or washing a PVDF with an N-alkyllactam which has been brought into contact with a solid acid substance, e.g. an ion exchange resin, or a mineral acid prior to distillation.

It is an object of the present invention to discover a method for purifying N-alkyllactams so that N-alkyllactams are obtainable which are suitable for the preparation of mixtures of N-alkyllactams and PVDF with an improved color quality.

It is a further object of the present invention to provide an improved mixture of PVDF and N-alkyllactams, which is obtainable by a process which is economically viable and which is easy to implement technologically and allows easy recycling and regeneration of the adsorbent. In particular it was an object of the present invention to avoid contacting N-alkyllactams with an acid because residual acid traces remaining in the N-alkyllactam after treatment may trigger corrosion and otherwise lead to quality fluctuations, which is problematic for the application of PVDF for battery binder.

We have accordingly found a process for purifying N-alkyllactam which comprises contacting N-alkyllactams with active carbon.

The N-alkyllactam used in the process according to the invention is preferably an N-alkyllactam of the general formula I

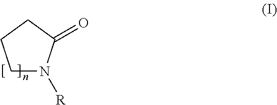

in which R is a
linear or branched, saturated aliphatic radical, preferably C₁₋₁₂-alkyl, such as methyl, ethyl, n-propyl, isopropyl, n-butyl, isobutyl, sec-butyl, tert-butyl, n-pentyl, isopentyl, sec-pentyl, neo-pentyl, 1,2-dimethylpropyl, n-hexyl, isohexyl, sec-hexyl, cyclopentylmethyl, n-heptyl, isoheptyl, cyclohexylmethyl, n-octyl, 2-ethylhexyl, n-nonyl, isononyl, n-decyl, isodecyl, n-undecyl, n-dodecyl, isododecyl, more preferably C₁₋₈-alkyl, such as methyl, ethyl, n-propyl, isopropyl, n-butyl, isobutyl, sec-butyl, tert-butyl, n-pentyl, n-hexyl, n-octyl and 2-ethylhexyl, most preferably C₁₋₄-alkyl, such as methyl, ethyl, n-propyl, isopropyl, n-butyl, isobutyl, sec-butyl, tert-butyl,
   or a
saturated cycloaliphatic radical having from 3 to 12 carbon atoms, preferably C₄₋₈-cycloalkyl, such as cyclobutyl, cyclopentyl, cyclohexyl, cycloheptyl and cyclooctyl, more preferably cyclopentyl and cyclohexyl,
and n is an integer from 1 to 5
and where the carbon atoms of the heterocyclic ring of the N-substituted lactam may bear from one to two substituents inert under the conditions, for example alkyl radicals, e.g. C₁₋₈-alkyl radicals, which are each independently preferably a C₁₋₈-alkyl radical, particularly a C₁₋₄-alkyl radical.

According to the invention R can also be H. Therefore the compound 2-Pyrrolidone is therefore also included in the definition of N-alkyllactam in the present invention.

Examples of C₁₋₈-alkyl radicals which may bear the carbon atoms of the heterocyclic ring of the N-substituted lactam are:
methyl, ethyl, n-propyl, isopropyl, n-butyl, isobutyl, sec-butyl, tert-butyl, n-pentyl, isopentyl and 2-ethylhexyl,
for example in 1,5-dimethyl-2-pyrrolidone and 1-ethyl-5-methyl-2-pyrrolidone.

Particular preference is given to using N-alkyllactams of the formula I

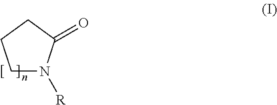

in which R is C₁₋₄-alkyl as described above and n is 1, 2 or 3, and where the carbon atoms of the heterocyclic ring of the N-substituted lactam may bear a C₁₋₄-alkyl radical, particularly methyl or ethyl radical.

Most preferred N-alkyllactams are N-Methyl-2-pyrrolidone (NMP) and N-Ethyl-2-pyrrolidone (NEP)

The N-alkyllactams used may have a purity of ≧90% by weight, preferably ≧95% by weight, more preferably ≧99% by weight.

The preparation of N-alkyllactams is known. N-alkylpyrrolidones can be effected, for example, by reacting gamma-butyrolactone (γ-BL) with monoalkylamines to release one equivalent of water, for example analogously to Ullmann's Encyclopedia of Industrial Chemistry, volume A22, 5th ed., p. 459 (1993) or analogously to DE-A-19 626 123 (BASF AG). N-alkylpyrrolidones can likewise be prepared from maleic anhydride or other dicarboxylic acid derivatives and monoethylamines in the presence of hydrogen and a hydrogenation catalyst, for example according to EP-A-745 598 (Bayer AG) or WO-A-02/102773 (BASF AG).

Other N-alkyllactams such as N-alkylpiperidones and N-alkylccaprolactams can likewise be prepared from the corresponding lactones by reacting with monoalkylamines, as described, for example, by Yakugaku Zasshi 71 (1951), 1341 (Susagawa et al.). In addition, these lactams can also be obtained by reacting oxynitriles with monomethylamines, as disclosed in DE-A-11 92 208 (BASF AG), or else elegantly by reacting lactams with monoalcohols or dialkyl ethers over acidic catalysts such as Al₂O₃, as described in Chem. Techn. 33 (1981), 193-196 (Wehner et al., VEB Leuna) or RO 137218 (Centrul de Cercetari pentru Fibre Chimice), or else with other alkylating agents such as dialkyl sulfates or alkyl halides under basic conditions, as described, for example, in J. Org. Chem. 29 (1964), pages 2748-2750 (Moriarty).

According to the invention carbon is used for the treatment of N-alkyllactams. The carbon useful in this process can be any conventional carbon or charcoal used as an absorbent. Carbon, activated carbon and charcoal are widely available commercially.

Suitable carbon can be used from a wide variety of sources. For example, carbon known as bituminous coal type and coconut shell type are well known in the art.

The shape of the carbon is not critical and can be in the form of any conventional shape such as powder, granular, pellet, or the like.

The average size of carbon used in this invention can vary widely, but finely powdered carbons are less desirable since they are difficult to separate from the N-alkyllactams and tend to cause plugging in a conventional continuous flow system. Any size carbon can be used which is capable of being supported in a bed without plugging, as is apparent to a skilled artisan.

Carbon can be rendered active using conventional procedures such as treatment with an inorganic acid.

For this purpose, any inorganic acid, or its solutions in water or organic solvents may be used. Suitable organic solvents include solvents in which the acid is miscible, such as alcohols and ethers which are readily removed by drying. Subsequent to being contacted with an inorganic acid, the carbon employed may be rinsed with either deionized water, then organic solvent, such as methanol. Following rinsing, the carbon is preferably dried (typically by heating the carbon), then rinsed with N-alkyllactam prior to using the carbon in the treatment process.

The surface area of the carbon can vary widely from about 200 to 4000 m²/g.

Preferred carbons should have a specific surface area of from 400 to 3 000 m²/g, in particular from 700 to 1 500 m²/g.

The pore size distribution of the carbon can vary widely from about 0.1 nm to 100 nm, preferably from 0.1 nm to 50 nm.

In a preferred embodiment the carbon has a peak in the pore size distribution in the range of 0.1 to 10 nm, preferably in the range of 1 nm to 8 nm and most preferably in the range of 1.5 nm to 4.5 nm.

If the carbon has a peak in the pore size distribution in the above specified range a further reduction of the color number can be realized and an N-alkyllactam is attained which is very stable against discoloration.

Examples of grades of activated carbon are those marketed under the following trade names: Carbo Tech PAK 1220, from Carbo Tech, Chemviron CAL, from Chemviron, CPG LF 1240, from Chemviron, F300, F400, from Chemviron.

The treatment process using carbon can be run either batch-wise, semi-continuous or in a continuous manner.

The process of the present invention can be carried out by adding the carbon to the respective batch and bringing the N-alkyllactam and carbon into intimate contact with one another, for example by stirring or shaking. Carbon is then preferably separated off.

The process of this invention can be conducted in any batch system suitably designed for such purpose as is apparent to a person skilled in the arts.

Contact time will vary depending on factors such as temperature, pressure, volume of N-alkyllactam to be treated, and the amount of N-alkyllactam relative to carbon. Typically, contact time is greater than 0.01 hour. Preferably, time is greater than 0.1 hour. Typically, time is less than 24 hours. Preferably, time is less than 18 hours, more preferably less than 8 hours. In batch mode, the amount of carbon is preferably at least one percent by weight relative to N-alkyllactam, more preferably the amount of carbon is greater than about 5 percent.

The batch can be stirred.

Pressure can be atmospheric, sub-atmospheric, or super atmospheric.

A pad of an inert gas such as dry nitrogen can be maintained over the batch.

In a continuous treatment process, the N-alkyllactam to be treated is contacted with one or more fixed beds of carbon.

Conventional treatment apparatus are useful for this purpose.

In a preferred embodiment of the present invention, the carbons are installed in the form of a fixed bed and the N-alkyllactam is passed over the fixed bed.

The carbon-adsorbents can also be in the form of a melt bed or a fluidized bed.

The preferred continuous embodiment is that in fixed beds in a carousel arrangement, in particular with regeneration.

The preferred semicontinuous embodiment is that in two alternately operated fixed beds.

The contact time varies depending on conditions and may be expressed in terms of flow rate over carbon. Typically, the flow rate is greater than about 0.4 ml of N-alkyllactam ml per liter of carbon per hour, and in one embodiment greater than about 4 ml of N-alkyllactam per liter of carbon per hour. Typically, the flow rate is usually no more than about 200 ml of N-alkyllactam per liter of carbon per hour, in one embodiment the flow rate is usually no more than about 200 ml of N-alkyllactam per liter of carbon per hour, and in another embodiment is about 20 ml of N-alkyllactam per liter carbon per hour.

Pressures are preferably sufficient to maintain liquid conditions. In continuous operation, the apparatus is usually equipped in a conventional manner so that effluent is free of carbon particles.

The temperature at which the treatment according to the present invention of the N-alkyllactam with carbons is carried out ranges from 0 to 100° C., preferably from 0 to 50° C., in particular from 10 to 40° C.

The activity of the carbon may decline over time. Therefore, the carbon may require regeneration as necessary as determined by routine experimentation and observation.

Conventional procedures can be employed for this purpose. A polar solvent may be used to flush the carbon. Likewise, the carbon can be heated to burn off deposits. In a preferred embodiment of the process of the present invention, exhausted carbon is regenerated, thus making reuse or recirculation in the process possible.

Regeneration both of the activated carbon can be carried out using strong mineral acids and strong caustic alkalis. Examples are HCl in various concentrations, for example 5 and 10%, and strong caustic alkalis, for example NaOH and KOH, in various concentrations, for example 5 and 10%.

Regeneration of carbon can be carried out continuously, semi-continuously or batchwise.

After treatment, the N-alkyllactam can be separated from carbon using conventional techniques such as filtration.

The N-alkyllactam obtainable by the process according to the invention are in particularly suited for the preparation of mixtures of N-alkyllactam and PVDF having an improved color quality.

The present invention also relates to mixtures comprising poly(vinylidene fluodride) (PVDF) and N-alkyllactam, wherein the N-alkyllactam has been purified by contacting the N-alkyllactam with an adsorbent.

The mixtures according to the invention comprise N-alkyllactam.

The preparation of N-alkyllactam suitable for being brought into contact with an adsorbent is described above.

Preferred N-alkyllactams are NMP and NEP. Especially NMP is preferred.

According to the invention, the N-alkyllactams have been purified by contacting the N-alkyllactam with an adsorbent.

The adsorbent can be any material known to a person skilled in the art to possess adsorbent properties.

In the context of the present invention, the term “adsorbent” excludes solid acid substances described in JP-A1-10310795.

Suitable adsorbents are described in U.S. Pat. No. 4,501,902, such as alkaline earth carbonates, alkaline earth hydroxides, alkaline earth oxides, and alumina.

In particular the adsorbents used for treating N-alkyllactam are carbon or active carbon, silica, magnesium silicate, such as magnesium silicate known under the trademark Ambosol®, diatomaceous earth, alumina or magnesia.

Aluminosilicates, zirconium silicates or zeolites are also suitable adsorbents.

Preferred adsorbents are carbon or active carbon, silica, magnesium silicate, such as magnesium silicate known under the trademark Ambosol®, diatomaceous earth, alumina or magnesia.

Especially preferred adsorbents are carbon or active carbon, magnesium silicate, alumina or magnesia and the most preferred adsorbent is carbon or active carbon.

The particularly most preferred adsorbent is active carbon.

Carbon, which may be used as an adsorbent in the mixtures according to the invention, has been described in detail above.

In a preferred embodiment of the invention, the adsorbents are present in the form of molecular sieves. Molecular sieve is a material containing pores of an essentially precise and uniform type. Molecular sieve preferably consist of aluminosilicate minerals, clays, porous glasses, microporous charcoals, zeolites, active carbons or synthetic compounds that have open structures through which small molecules can diffuse. Further description of various molecular sieves may be found in Kirk-Othmer, Encyclopedia of Chemical Technology, 3d. ed., vol. 16, pp. 811-853 (2004), incorporated herein by reference.

Preferred molecular sieves are zeolites and aluminosilicates

The purification of the N-alkyllactam with the adsorbent is brought about by contacting the N-alkyllactam with the adsorbent.

The purification of N-alkyllactam by contacting the N-alkyllactam with an adsorbent can be run either batch-wise, semi-continuous or in a continuous manner.

The process of the present invention can be carried out by adding the adsorbent/adsorbents to the respective batch and bringing the N-alkyllactam and the adsorbent into intimate contact with one another, for example by stirring or shaking.

The adsorbent is then preferably separated off.

The process of this invention can be conducted in any batch system suitably designed for such purpose as is apparent to a person skilled in the arts.

Contact time will vary depending on factors such as temperature, pressure, volume of N-alkyllactam to be treated, and the amount of N-alkyllactam relative to the adsorbent. Typically, contact time is greater than 0.01 hour. Preferably, time is greater than 0.1 hour. Typically, time is less than 24 hours. Preferably, time is less than 18 hours, more preferably less than 8 hours. In batch mode, the amount of adsorbent is preferably at least one percent by weight relative to N-alkyllactam, more preferably the amount of adsorbent is greater than about 5 percent.

The batch can be stirred.

Pressure can be atmospheric, sub-atmospheric, or super atmospheric.

A pad of an inert gas such as dry nitrogen can be maintained over the batch.

In a continuous treatment process, the N-alkyllactam to be treated is contacted with one or more fixed beds of adsorbent.

Conventional treatment apparatus are useful for this purpose.

In a preferred embodiment of the present invention, the adsorbents are installed in the form of a fixed bed and the N-alkyllactam is passed over the fixed bed.

The adsorbents can also be in the form of a melt bed or a fluidized bed.

The preferred continuous embodiment is that in fixed beds in a carousel arrangement, in particular with regeneration.

The preferred semicontinuous embodiment is that in two alternately operated fixed beds.

The contact time varies depending on conditions and may be expressed in terms of flow rate over adsorbent. Typically, the flow rate is greater than about 0.4 ml of N-alkyllactam ml per liter of adsorbent per hour, and in one embodiment greater than about 4 ml of N-alkyllactam per liter of adsorbent per hour. Typically, the flow rate is usually no more than about 200 ml of N-alkyllactam per liter of adsorbent per hour, in one embodiment the flow rate is usually no more than about 200 ml of N-alkyllactam per liter of adsorbent per hour, and in another embodiment is about 20 ml of N-alkyllactam per liter adsorbent per hour.

Pressures are preferably sufficient to maintain liquid conditions. in continuous operation, the apparatus is usually equipped in a conventional manner so that effluent is free of adsorbent.

The temperature at which the treatment according to the present invention of the N-alkyllactam with adsorbents is carried out ranges from 0 to 100° C., preferably from 0 to 50° C., in particular from 10 to 40° C.

The activity of the adsorbent may decline over time. Therefore, the adsorbent may require regeneration as necessary as determined by routine experimentation and observation.

Conventional procedures can be employed for this purpose. A polar solvent may be used to flush the adsorbent. Likewise, the adsorbent can be heated to burn off deposits.

In a preferred embodiment of the process of the present invention, exhausted adsorbent is regenerated, thus making reuse or recirculation in the process possible. Regeneration of adsorbent can be carried out continuously, semi-continuously or batchwise.

After treatment, the N-alkyllactam can be separated from adsorbent using conventional techniques such as filtration.

In a preferred embodiment adsorption and filtration may be conducted in a single process step by filtering the N-alkyllactam with a volume bulk filter with a positive zeta potential. Such volume bulk filter are filter modules based on cellulose with additives such as diatomaceous earth, perlites, synthetic polymers (modified nylon, polyvinylpyridine) and active carbon or carbon. Such filter are obtainable from the company Seitz (K- und T-Series).

In another preferred embodiment the N-alkyllactam is brought into contact with both carbon, preferably active carbon, and molecular sieve in combination. Contact can be either sequentially or simultaneously. Preferably the N-alkyllactam is first brought into contact with molecular sieve and afterwards brought into contact with carbon, for example by passing over the N-alkyllactam over a bed of molecular sieve and then over a bed of carbon. Purifying N-alkyllactam with carbon and molecular sieve in combination leads to a further decrease of the color number and renders N-alkyllactam, which is very stable against discoloration.

The mixtures of the present invention comprise PVDF.

PVDF is commercially available, e.g. as Kynar® from Arkema, Dyneon® from Dyneon and Solet® from Solvay S.A. and KF-Polymer® from Kureha.

Methods of preparation are disclosed for instance in Kirk-Othmer Encyclopedia of Chemical Technology (Kirk-Othmer Encyclopedia of Chemical Technology, “Fluorine-Containing Polymers, Poly(vinylidene fluoride), Electronic Edition, Last updated: 17 Oct. 2008, John Wiley & Sons, Inc.).

The content of PVDF in the mixtures according to the invention is between 1 and 95% by weight based on the combined weight of N-alkyllactams and, preferably between 1 and 80% by weight, most preferably between 1 and 60% by weight, in particularly between 1 and 50% by weight based on N-alkyllactams.

The present invention also relates to a process for preparing a mixture with improved color quality comprising PVDF by bringing into contact PVDF and N-alkyllactam, which has been purified by contacting the N-alkyllactam with an adsorbent.

The mixtures according to the invention may be prepared by bringing PVDF and N-alkyllactam into contact, most preferably by stirring the mixture e.g. in a stirred tank reactor. Suitable stirrers, e.g. planetary stirrers, and vessels for preparing the mixtures according to the invention are known to a person skilled in the arts.

The temperature range at which the components of the mixture are brought into contact is in the usually in the range of 0 to 200° C., preferably at 0 to 100° C. and most preferably at 10 to 100° C. and most preferably at 10° to 50° C., in particularly at ambient temperatures.

The duration of mixing the components to obtain a homogeneous solution depends on the concentration of PVDF and the temperature. Usually the duration of mixing is between 1 minutes and 24 hours, preferably between 5 minutes and 12 hours, most preferably between 10 minutes and 6 hours and in particularly between 15 minutes and 2 hours.

The components of the mixture can be brought into contact under atmospheric conditions or under inert conditions, e.g. under a nitrogen atmosphere. Most preferably the mixture is mixed under inert conditions.

The mixtures of the present invention may also contain other components, such as cosolvents, fillers, processing aids, other polymers, salts, which are required for the specific application.

The mixtures can be processed directly after mixing or they can be stored. Usually the mixtures are stored at ambient temperature.

The mixtures according to the invention have an improved color quality. Upon the addition of PVDF to the N-alkyllactam, the mixtures according to the invention do not turn brown or black but keep a light clear color.

The Iodine color numbers determined according to DIN 6162 of the mixtures of the present invention are preferably less than 500, more preferably less than 300, even more preferably less than 100 and in particularly less than 50.

The mixtures of the present invention can be used in processing PVDF for applications in which improved color is a quality requirement.

In particularly the mixtures according to the invention can be used for making PVDF-films and/or membranes which show an improved color quality.

Preferably, the mixtures according to the invention are also used for the production of battery binder.

The present invention therefore also relates to a method of processing PVDF using mixtures according to the invention.

Membranes of improved color quality may be prepared according to the method disclosed by Grandine et al. (U.S. Pat. No. 4,203,847) or Benzinger et. al (U.S. Pat. No. 4,384,047), which are herein incorporated by reference, processing the mixtures of the present invention instead of the solvents disclosed within these references.

Such membranes and films are required in the fabrication of light emitting diodes fuel cells and in particularly lithium batteries for the preparation of the electrode material or as a solid electrolyte. Methods for preparing electrode material and electrolytes based on PVDF are disclosed for example in WO-A1-01/65616, EP-A1-0567015, U.S. Pat. No. 5,900,183, U.S. Pat. No. 5,962,167 EP-A1-0793286, WO-A1-01/82403 and U.S. Pat. No. 6,510,042.

The advantage of the present invention is that a mixture has been found which is suitable for the processing of PVDF for applications in which an improved color is of importance. This mixture is easily obtainable. These mixtures may be used for the fabrication of membranes and films used in electronic applications, in which color quality and the impurities associated with discoloration are undesirable.

The process according to the invention for purifying N-alkyllactams is economically viable and results in N-alkyllactams with may be used for the production of solutions of PVDF and N-alkyllactams with an improved color quality.

The application is illustrated in the following examples:

### EXAMPLE 1

100 ml of NMP (origin BASF) were brought in contact with 5 g of active carbon (Carbo-raffin® from JapanEnviroChemicals Ltd.). Active carbon was separated from NMP by filtering NMP through a paper filter.

10 weight-% PVDF (Kureha KF W1100) were added to the filtered NMP at room temperature under a blanket of nitrogen. The mixture was stirred while the temperature was slowly increased to 80° C. until the polymer dissolved.

After the polymer was solved, the temperature was reduced to room temperature and the iodine color number of the PVDF-solution was measured. The iodine color number was determined according to DIN 6162 to be 1.

### COMPARISON EXAMPLE 1

10 weight-% PVDF (Kureha KF W1100) were added to untreated NMP at room temperature under a blanket of nitrogen. The mixture was stirred while the temperature was slowly increased to 80° C. until the polymer dissolved.

After the polymer was solved, the temperature was reduced to room temperature and the iodine color number of the PVDF-solution was measured. The iodine color number was determined according to DIN 6162 to be 1100.

### EXAMPLE 2

100 ml of NEP (origin BASF) were brought in contact with 5 g of active carbon (Carbo-raffin® from JapanEnviroChemicals Ltd.). Active carbon was separated from NMP by filtering NMP through a paper filter.

10 weight-% PVDF (Kureha KF W1100) were added to the filtered NEP at room temperature under a blanket of nitrogen. The mixture was stirred while the temperature was slowly increased to 80° C. until the polymer dissolved.

After the polymer was solved, the temperature was reduced to room temperature and the iodine color number of the PVDF-solution was measured. The iodine color number was determined according to DIN 6162 to be 1.

### COMPARISON EXAMPLE 2:

10 weight-% PVDF (Kureha KF W1100) were added to untreated NEP at room temperature under a blanket of nitrogen. The mixture was stirred while the temperature was slowly increased to 80° C. until the polymer dissolved.

After the polymer was solved, the temperature was reduced to room temperature and the iodine color number of the PVDF-solution was measured. The iodine color number was determined according to DIN 6162 to be 1100.

1.-20. (canceled)

21. A process for purifying an N-alkyllactam of formula I 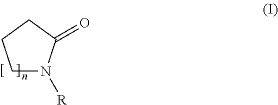
in which R is a
linear or branched, saturated aliphatic C₁₋₁₂-alkyl radical, or a saturated cycloaliphatic radical having from 3 to 12 carbon atoms,
and n is an integer from 1 to 5,
and where the carbon atoms of the heterocyclic ring of the N-substituted lactam optionally contain from one to two C₁₋₈-alkyl radicals,
which comprises contacting the N-alkyllactam with carbon and separating the N-alkyllactam from carbon.

22. A process for preparing a mixture with an iodine color number measured according to DIN 6162 of less than 500 comprising poly(vinylidenefluoride) (PVDF) and either 2-pyrrolidone or N-alkyllactam of formula I, 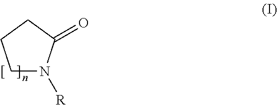
in which R is a
linear or branched, saturated aliphatic C₁₋₁₂-alkyl radical, or a saturated cycloaliphatic radical having from 3 to 12 carbon atoms,
and n is an integer from 1 to 5,
and where the carbon atoms of the heterocyclic ring of the N-substituted lactam optionally contain from one to two C₁₋₈-alkyl radicals,
comprising:
a) purifying the 2-pyrrolidone or the N-alkyllactam by contacting the 2-pyrrolidone or the N-alkyllactam with an adsorbent; and
b) mixing the purified 2-pyrrolidone or the N-alkyllactam obtained in step a) with PVDF, and
wherein the adsorbent comprises carbon.

23. The process according to claim 22, wherein in step b) the content of PVDF is 1 to 95 percent by weight of N-alkyllactam or 2-pyrrolidone.

24. The process according to claim 22, wherein in step b) the content of PVDF is 1 to 50 percent by weight based on N-alkyllactam or 2-pyrrolidone.

25. The process according to claim 22, wherein the N-alkyllactam is N-Methyl-pyrrolidone (NMP) and/or N-Ethyl-pyrrolidone (NEP).

26. The process according to claim 22, wherein the adsorbent has a peak in pore size distribution in the range from 1.5 nm to 4.5 nm.

27. The process according to claim 22, wherein the adsorbent comprises a combination of carbon and molecular sieve.

28. The process according to claim 22, wherein in step a) the adsorbent is brought in contact with PVDF at a temperature of 15 to 100° C. for a duration of 5 minutes to 24 hours.

29. A process for processing PVDF-mixtures with a iodine color number measured according to DIN 6162 of less than 500 comprising PVDF and either 2-pyrrolidone or N-alkyllactam of formula I, 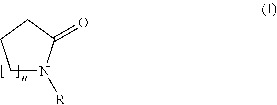
in which R is a
linear or branched, saturated aliphatic C₁₋₁₂-alkyl radical, or a saturated cycloaliphatic radical having from 3 to 12 carbon atoms,
and n is an integer from 1 to 5,
and where the carbon atoms of the heterocyclic ring of the N-substituted lactam optionally contain from one to two C₁₋₈-alkyl radicals,
comprising
a) purifying the 2-pyrrolidone or the N-alkyllactam by contacting the 2-pyrrolidone or the N-alkyllactam with an adsorbent;
b) mixing the purified 2-pyrrolidone or the N-alkyllactam obtained in step a) with poly(vinylidenefluoride) (PVDF); and
c) processing the mixture obtained in step b); and
wherein the adsorbent is carbon.

30. Process for the manufacture of a film, membrane, coating or electronic application PVDF from mixtures with a iodine color number measured according to DIN 6162 of less than 500 comprising PVDF and either 2-pyrrolidone or N-alkyllactam of formula I, 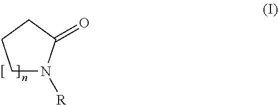
in which R is a
linear or branched, saturated aliphatic C₁₋₁₂-alkyl radical, or a saturated cycloaliphatic radical having from 3 to 12 carbon atoms,
and n is an integer from 1 to 5,
and where the carbon atoms of the heterocyclic ring of the N-substituted lactam optionally contain from one to two C₁₋₈-alkyl radicals,
comprising
a) purifying the 2-pyrrolidone or the N-alkyllactam by contacting the 2-pyrrolidone or the N-alkyllactam with an adsorbent;
b) mixing the purified 2-pyrrolidone or the N-alkyllactam obtained in step a) with poly(vinylidenefluoride) (PVDF); and
c) using the mixture obtained in step b) for the manufacture of the film, membrane, coating or electronic application; and
wherein the adsorbent is carbon.

31. The process according to claim 30, wherein the electronic application is a lithium battery.
